(19) United States
(12) Patent Application Publication
(10) Pub. No.: US 2024/0325355 A1
(43) Pub. Date: Oct. 3, 2024

(54) REDUCED DOSE METAXALONE FORMULATIONS

(71) Applicant: Primus Pharmaceuticals, Inc., Scottsdale, AZ (US)

(72) Inventors: Mukteeshwar Gande, Denville, NJ (US); Robert M. Levy, Cave Creek, AZ (US); Sam G. Salamone, Scottsdale, AZ (US); Trevor T. Hall, Scottsdale, AZ (US)

(73) Assignee: Primus Pharmaceuticals, Inc., Scottsdale, AZ (US)

(21) Appl. No.: 18/614,759

(22) Filed: Mar. 25, 2024

Related U.S. Application Data
(63) Continuation-in-part of application No. 18/409,208, filed on Jan. 10, 2024.
Application No. 18/409,208 is a continuation of application No. 18/197,100, filed on May 14, 2023, now Pat. No. 11,918,559.
Application No. 18/197,100 is a continuation of application No. 16/524,952, filed on Jul. 29, 2019, now abandoned.
(60) Provisional application No. 62/866,356, filed on Jun. 25, 2019.

(51) Int. Cl. A61K 31/421; A61K 9/00; A61K 9/20; A61K 9/48

(52) U.S. Cl. CPC A61K 31/421; A61K 9/0053; A61K 9/2013; A61K 9/4858

(57) ABSTRACT

Oral dosage forms of metaxalone having improved bioavailability in the fed and fasted states, including dosage forms that employ a reduced dose based on such improved bioavailability.

### FIELD OF THE INVENTION

The present invention relates to oral dosage forms of metaxalone having improved bioavailability in the fed and fasted states, reduced food effect and the dose reduction attendant to such improved bioavailability.

### BACKGROUND OF THE INVENTION

Metaxalone (Skelaxin®), known chemically as 5-[(3,5-dimethylphenoxy)methyl]-2-oxazolidinone, has the following free-form chemical structure:

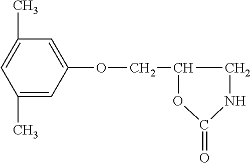 [SMILES: Cc1cc(C)cc(OCC2CNC(=O)O2)c1]

Skelaxin is indicated as an adjunct to rest, physical therapy, and other measures for the relief of discomforts associated with acute, painful musculoskeletal conditions. The mode of action of this drug has not been clearly identified but may be related to its sedative properties. Metaxalone does not directly relax tense skeletal muscles in man. The commercially available tablet contains: metaxalone, 400 and 800 mg along with inert compression tableting excipients.

Preparation of metaxalone is described in Lunsford et al., J. Am. Chem. Soc. 82, 1166 (1960) and U.S. Pat. No. 3,062,827 to Lunsford (Nov. 6, 1962, Assignee A. H. Robins), which is incorporated herein in its entirety by reference. The '827 patent discloses the compound and related species as anticonvulsants and antispasmodics; however, these activities have not been borne out by clinical experience.

The FDA-approved prescribing information for Skelaxin® indicates that the drug suffers from a significant food effect. In particular, the prescribing information reports for an 800 mg dose that “[c]ompared to fasted conditions, the presence of a high fat meal at the time of drug administration increased Cₘₐₓ by 193.6% and increased AUC (AUC₀₋ₜ, AUC_{∞}) by 146.4% and 142.2%, respectively. Time-to-peak concentration (Tₘₐₓ) was also delayed (4.9 h versus 3.0 h) and terminal half-life was decreased (4.2 h versus 8.0 h) under fed conditions compared to fasted conditions. This food effect generally limits the administration of the drug to the fasted state (taking on empty stomach) and significant impairs the utility of the drug.

### SUMMARY OF INVENTION

The inventors have unexpectedly discovered that the food effect associated with prior art metaxalone formulations can be avoided using a formulation that meets specified dissolution criteria in 0.5% Sodium Lauryl Sulfate (“SLS”) and/or Fasted State Simulated Intestinal Fluid (“FaSSIF”) (pH 6.5). Thus, in a first principal embodiment the invention provides a solid oral pharmaceutical formulation comprising micronized and non-micronized crystalline free-form metaxalone and one or more pharmaceutically acceptable excipients wherein: (a) a 640 mg tablet or capsule of said formulation releases at least ˜50 wt %, ˜55 wt %, ˜60 wt %, ˜65 wt %, or ˜70 wt % of its metaxalone in 60 minutes when tested in 900 mL 0.5% SLS in water in a USP Apparatus Type 2 (paddle) at 100 rpm and 37±0.5° C.; and/or (b) a 100 mg tablet or capsule of said formulation releases at least ˜65 wt %, ˜70 wt %, ˜75 wt %, or ˜80 wt % of its metaxalone in 300 minutes when tested in 900 mL of fasted state simulated intestinal fluid in a USP Apparatus Type 2 (paddle) at 50 rpm and 37±0.5° C.

This food effect associated with prior art formulations of metaxalone can also be overcome using a formulation that meets specified dissolution criteria in pH 4.5 acetate buffer dissolution medium and pH 6.0 phosphate buffer dissolution medium. Thus, in a second principal embodiment the invention provides a solid oral pharmaceutical formulation comprising micronized and non-micronized crystalline free-form metaxalone and one or more pharmaceutically acceptable excipients wherein (a) a 640 mg tablet or capsule of said formulation releases no more than ˜65 wt %, ˜60 wt %, ˜55 wt %, ˜50 wt %, or ˜45 wt % of its metaxalone at 90 minutes when tested in 900 mL of a pH 4.5 acetate buffer dissolution medium in a USP Apparatus Type 2 (paddle) at 100 rpm and 37±0.5° C.; and/or (b) a 640 mg tablet or capsule of said formulation releases no more than ˜65 wt %, ˜60 wt %, ˜55 wt %, ˜50 wt %, or ˜45 wt % of its metaxalone at 90 minutes when tested in 900 mL of a pH 6.0 phosphate buffer dissolution medium in a USP Apparatus Type 2 (paddle) at 100 rpm and 37±0.5° C.

The invention further provides formulations capable of achieving the dissolution criteria in the first and second principal embodiments, and thereby overcoming the food effect of prior art metaxalone formulations. Thus, in a third principal embodiment the invention provides a solid oral pharmaceutical formulation selected from an oral dosage form (e.g., a tablet and a capsule) comprising from ˜40 to ˜80 wt % micronized particles of crystalline free-form metaxalone and from ˜20 to ˜60 wt % non-micronized particles of crystalline free-form metaxalone, wherein (a) ˜90 w/w % of the micronized particles of metaxalone are smaller than ˜500, ˜350, ˜200, ˜100, ˜75, or ˜50 microns when tested according to the Malvern Method; and (b) less than ˜10 w/w %, ˜5 w/w %, or ˜2 w/w % of the non-micronized particles are retained on a #30 Sieve, and at least ˜25 w/w %, ˜35 w/w %, or ˜45 w/w % of the non-micronized particles of metaxalone are retained on a #120 Sieve when tested by the Sieve Method.

In a fourth principal embodiment the invention provides a solid oral pharmaceutical formulation (i.e., dosage form) selected from a tablet and a capsule comprising (a) 640 weight parts micronized and non-micronized crystalline free-form metaxalone; and (b) from 10 to 30 weight parts propylene glycol alginate.

In a fifth principal embodiment the invention provides a method of treating musculoskeletal pain comprising administering to a patient in need thereof 640 mg of micronized and non-micronized crystalline free-form metaxalone in the formulation of any of the principal embodiments or subembodiments of the present invention, in the fasted or fed state, preferably in the fasted state.

Additional advantages of the invention are set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

[Incorporate manufacturing application Ser. No. 18/198,266 by reference]

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is a graphical depiction of the rate of release of metaxalone from prior art Skelaxin® 800 mg tablets (diamonds) and 640 mg micronized and non-micronized crystalline free-form metaxalone tablets (squares) (manufactured according to the current invention, in sodium lauryl sulfate dissolution medium, as described in Example 5.

### DETAILED DESCRIPTION

### Definitions and Use of Terms

When the singular forms “a,” “an” and “the” or like terms are used herein, they will be understood to include plural referents unless the context clearly dictates otherwise. Thus, for example, reference to “an excipient” includes mixtures of two or more such excipients, and the like. The word “or” or like terms as used herein means any one member of a particular list and also includes any combination of members of that list.

When used herein the term “about” or “ca.” will compensate for variability allowed for in the pharmaceutical industry and inherent in pharmaceutical products, such as differences in product strength and bioavailability due to manufacturing variations and time-induced product degradation. The term allows for any variation which in the practice of pharmaceuticals would allow the product being evaluated to be considered pharmaceutically equivalent or bioequivalent, or both if the context requires, to the recited strength of a claimed product. It will be understood that all numeric values expressed in this document can be prefaced by the term “about.”

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality of components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or specifying particular numerical values, it will be understood that a separate range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

As used herein, “therapeutically effective amount” refers to an amount sufficient to elicit the desired biological response. The therapeutically effective amount or dose will depend on the age, sex and weight of the patient, and the current medical condition of the patient. The skilled artisan will be able to determine appropriate dosages depending on these and other factors in addition to the present disclosure.

“Pharmaceutically acceptable” means that which is useful in preparing a pharmaceutical composition that is generally safe, non-toxic and neither biologically nor otherwise undesirable and includes that which is acceptable for human or veterinary pharmaceutical use. “Pharmaceutically acceptable salts” means salts that are pharmaceutically acceptable, as defined above, and which possess the desired pharmacological activity.

When a dose of a drug or its pharmaceutically acceptable salt is described herein, it will be understood that the dose is based on the weight of the free base, excluding any hydrates or solvates thereof, unless the description states that the dose is based on the weight of the salt, hydrate or solvate.

Throughout the patent application, wherever an analysis by a method prescribed in the United States Pharmacopoeia (“USP”) is prescribed, it will be understood that the analysis is performed in accordance with the USP volume in effect on Jan. 1, 2019. It will also be understood that the test need not have been performed, but that the test, if performed, would yield the claimed result. In like manner, any terms not otherwise defined herein can be defined by reference to the USP volume in effect on Jan. 1, 2019.

The term fasted state simulated intestinal fluid or “FaSSIF” refers to the following dissolution media at pH 6.5, as described in Table II by Klein S. The AAPS Journal, Vol. 12, No. 3, September 2010.

| | | |
|---|---|---|
| sodium taurocholate | 3 | mM |
| lecithin | 0.75 | mM |
| NaH₂PO₄ | 4.438 | g |
| NaCl | 6.186 | g |

| | |
|---|---|
| NaOH | qs ad to pH 6.5 |
| deionized water | qs ad to 1 L |
| osmolality (mOsmol/kg) | ~270 |
| buffer capacity (mEq/pH/L) | ~12 |
| surface tension (mN/m) | 54 |

The Sieve Method refers to the method for particle size analysis described in American Society for Testing and Materials (ASTM) standard C 136 (in effect on Jan. 1, 2019). In the method a representative weighed sample is poured into the top sieve which has the largest screen openings. Each lower sieve in the column has smaller openings than the one above. At the base is a round pan, called the receiver. The column is typically placed in a mechanical shaker. such as the sonic sifter available from Endecotts (London, UK). See Endecotts website at https://www.endecotts.com/products/sieve-shakers/sonic-sifter/product-specifications/. The shaker shakes the column, usually for some fixed amount of time. After the shaking is complete the material on each sieve is weighed. The mass of the sample of each sieve is then divided by the total mass to give a percentage retained on each sieve. The size of the average particle on each sieve is then analyzed to get a cut-off point or specific size range, which is then captured on a screen.

As used herein, the No. 30 mesh is a medium size U.S. Standard mesh size with a 0.0234″ (600 μm) nominal sieve opening with a typical wire diameter of 0.4 mm. Die-formed frames assure a proper fit, superior quality and performance, guaranteeing that the American Society for Testing Materials (ASTM) specifications are always met.

As used herein, the No. 120 mesh is a fine size U.S. Standard mesh size with a 0.0049″ (125 μm) nominal sieve opening with a typical wire diameter of 0.09 mm. Die-formed frames assure a proper fit, superior quality and performance, guaranteeing that the American Society for Testing Materials (ASTM) specifications are always met.

As used herein, the No. 325 mesh is a fine size U.S. Standard mesh size with a 0.0017″ (45 μm) nominal sieve opening with a typical wire diameter of 0.032 mm. Die-formed frames assure a proper fit, superior quality and performance, guaranteeing that the American Society for Testing Materials (ASTM) specifications are always met.

### Principal Embodiments

The invention is described herein in terms of principal embodiments and subembodiments. It will be understood that each of the subembodiments can modify any of the principal embodiments, unless such modification is logically inconsistent or expressly disallowed in this document. It will be further understood that the principal embodiments can be combined in any manner, and that the subembodiments can be combined in any manner to further modify any of the principal embodiments, unless such combination is logically inconsistent or expressly disallowed in this document.

In a first principal embodiment the invention provides a solid oral pharmaceutical formulation (i.e., an oral dosage form) comprising micronized and non-micronized crystalline free-form metaxalone and one or more pharmaceutically acceptable excipients wherein: (a) a 640 mg tablet or capsule of said formulation releases at least ˜50 wt %, ˜55 wt %, ˜60 wt %, ˜65 wt %, or ˜70 wt % of its micronized and non-micronized metaxalone in 60 minutes when tested in 900 mL 0.5% SLS in water in a USP Apparatus Type 2 (paddle) at 100 rpm and 37±0.5° C.; and/or (b) a 100 mg tablet or capsule of said formulation releases at least ˜65 wt %, ˜70 wt %, ˜75 wt %, or ˜80 wt % of its metaxalone in 300 minutes when tested in 900 mL of fasted state simulated intestinal fluid in a USP Apparatus Type 2 (paddle) at 50 rpm and 37±0.5° C. (See, https://www.agilent.com/en/product/dissolution-testing/dissolution-accessories-supplies/dissolution-paddles-baskets-sinkers/dissolution-paddles?gad_source=1&gclid=Cj0KCQiAgK2qBhCHARIsAGACuznZOLrbnGLVZ0M2ZtjwU UQEEu3gwjZ9kmuw021yojPHTOYvTMFqynlaAvFrEALw_wcB&gclsrc=aw.ds).

In a second principal embodiment the invention provides a solid oral pharmaceutical formulation (i.e., an oral dosage form) comprising micronized and non-micronized crystalline free-form metaxalone and one or more pharmaceutically acceptable excipients wherein (a) a 640 mg tablet or capsule of said formulation releases no more than ˜65 wt %, ˜60 wt %, ˜55 wt %, ˜50 wt %, or ˜45 wt % of its metaxalone at 90 minutes when tested in 900 mL of a pH 4.5 acetate buffer dissolution medium in a USP Apparatus Type 2 (paddle) at 100 rpm and 37±0.5° C.; and/or (b) a 640 mg tablet or capsule of said formulation releases no more than ˜65 wt %, ˜60 wt %, ˜55 wt %, ˜50 wt %, or ˜45 wt % of its metaxalone at 90 minutes when tested in 900 mL of a pH 6.0 phosphate buffer dissolution medium in a USP Apparatus Type 2 (paddle) at 100 rpm and 37±0.5° C.

In a third principal embodiment the invention provides a solid oral pharmaceutical formulation (i.e., an oral dosage form) selected from a tablet and a capsule comprising from ˜40 to ˜80 wt % micronized crystalline free-form particles of metaxalone and from ˜20 to ˜60 wt % non-micronized particles of crystalline free-form metaxalone, wherein (a) ˜90 w/w % of the micronized particles of metaxalone are smaller than ˜500, ˜350, ˜200, ˜100, ˜75, or ˜50 microns when tested according to the Malvern Method; and (b) at least ˜20 w/w %, ˜25 w/w %, ˜30 w/w %, or ˜35 w/w % of the non-micronized particles of metaxalone are retained on a #120 Sieve when tested by the Sieve Method.

In a fourth principal embodiment the invention provides a solid oral pharmaceutical formulation (i.e., an oral dosage form) selected from a tablet and a capsule comprising (a) 640 weight parts micronized and non-micronized crystalline free-form metaxalone; and (b) from ˜10 to ˜30 weight parts propylene glycol alginate.

In a fifth principal embodiment the invention provides a method of treating musculoskeletal pain comprising administering to a patient in need thereof 640 mg of micronized and non-micronized crystalline free-form metaxalone in the formulation of any of the principal embodiments or subembodiments or other embodiments set forth herein of the present invention, in the fasted or fed state.

### Subembodiments and Other Embodiments

The invention can further be defined in terms of various subembodiments and other embodiments, each of which can modify any of the principal embodiments singularly or in any combination.

In various subembodiments of the present invention a 640 mg tablet or capsule of the formulation can release at least ˜50 wt %, ˜55 wt %, ˜60 wt %, ˜65 wt %, or ˜70 wt % of its micronized and non-micronized crystalline free-form metaxalone in 60 minutes when tested in 900 mL 0.5% SLS in water in a USP Apparatus Type 2 (paddle) at 100 rpm and 37±0.5° C.

In a particularly preferred subembodiment a 640 mg tablet or capsule of said formulation releases at least ˜60 wt % of its micronized and non-micronized crystalline free-form metaxalone in 60 minutes when tested in 900 mL 0.5% SLS in water in a USP Apparatus Type 2 (paddle) at 100 rpm and 37±0.5° C.; and

In other subembodiments of the present invention a 100 mg tablet or capsule of said formulation releases at least ˜65 wt %, ˜70 wt %, ˜75 wt %, or ˜80 wt % of its micronized and non-micronized crystalline free-form metaxalone in 300 minutes when tested in 900 mL of fasted state simulated intestinal fluid in a USP Apparatus Type 2 (paddle) at 50 rpm and 37±0.5° C.

In a particularly preferred subembodiment a 100 mg tablet or capsule of said formulation releases at least ˜75 wt % of its micronized and non-micronized crystalline free-form metaxalone in 300 minutes when tested in 900 mL of fasted state simulated intestinal fluid in a USP Apparatus Type 2 (paddle) at 50 rpm and 37±0.5° C.

In another subembodiment a 640 mg tablet or capsule of said formulation releases no more than ˜65 wt %, ˜60 wt %, ˜55 wt %, ˜50 wt %, or ˜45 wt % of its micronized and non-micronized crystalline free-form metaxalone at 90 minutes when tested in 900 mL of a pH 4.5 acetate buffer dissolution medium in a USP Apparatus Type 2 (paddle) at 100 rpm and 37±0.5° C.

In a particularly preferred subembodiment a 640 mg tablet or capsule of said formulation releases no more than ˜65 w/w % of its micronized and non-micronized crystalline free-form metaxalone at 90 minutes when tested in 900 mL of a pH 4.5 acetate buffer dissolution medium in a USP Apparatus Type 2 (paddle) at 100 rpm and 37±0.5° C.

In still another subembodiment a 640 mg tablet or capsule of said formulation releases no more than ˜65 wt %, ˜60 wt %, ˜55 wt %, ˜50 wt %, or ˜45 wt % of its micronized and non-micronized crystalline free-form metaxalone at 90 minutes when tested in 900 mL of a pH 6.0 phosphate buffer dissolution medium in a USP Apparatus Type 2 (paddle) at 100 rpm and 37±0.5° C.

In a particularly preferred subembodiment a 640 mg tablet or capsule of said formulation releases no more than ˜65 w/w % of its micronized and non-micronized crystalline free-form metaxalone at 90 minutes when tested in 900 mL of a pH 6.0 phosphate buffer dissolution medium in a USP Apparatus Type 2 (paddle) at 100 rpm and 37±0.5° C.

The formulations of the present invention can also be defined in terms of micronized and non-micronized crystalline free-form metaxalone particles having a particle size or particle size distribution. In one subembodiment the formulation comprises from ˜40 to ˜80 wt % micronized crystalline free-form particles of metaxalone and from ˜20 to ˜60 wt % non-micronized crystalline free-form particles of metaxalone. In one particular subembodiment the formulation comprises from ˜30 to ˜50 wt % or from ˜35 to ˜45 wt % micronized crystalline free-form particles of metaxalone and from ˜50 to ˜70 wt % or from ˜55 to ˜65 wt % non-micronized crystalline free-form particles of metaxalone.

In one subembodiment, when the formulation is characterized based on metaxalone particle size, at least ˜50 w/w %, ˜70 w/w %, or ˜90 w/w % of the micronized particles of crystalline free-form metaxalone are smaller than ˜200, ˜100, or ˜75 microns when tested according to the Malvern Method (i.e. laser diffraction). Alternatively or in addition, at least ˜30 w/w %, ˜40 w/w %, or ˜50 w/w % of the micronized crystalline free-form metaxalone particles are less than ˜50, ˜30, or ˜20 microns. when tested according to the Malvern Method.

In another subembodiment, no more than ˜10 w/w %, ˜5 w/w % or ˜2 w/w % of the non-micronized crystalline free-form metaxalone particles are retained on a #30 Sieve, and at least ˜15 w/w %, ˜25 w/w %, ˜35 w/w %, or ˜45 w/w % of the non-micronized crystalline free-form particles of metaxalone are retained on a #120 Sieve when tested by the Sieve Method. In a preferred subembodiment, at least ˜10 w/w % or ˜20 w/w % of the non-micronized crystalline free-form particles are in addition retained on a #325 Sieve when tested by the Sieve Method.

In still further embodiments the formulations of the present invention are defined based on the ingredients used to make the formulation. Thus, in one subembodiment, the formulations of the present invention comprise 640 weight parts metaxalone and from ˜10 to ˜30 weight parts or from ˜15 to ˜25 weight parts propylene glycol alginate.

In still further subembodiments of formulations containing propylene glycol alginate, the formulations comprise from ˜20 to ˜35 weight parts or from ˜24 to ˜31 weight parts lactose monohydrate; from ˜10 to ˜30 or from ˜15 to ˜25 weight parts alginic acid; from ˜40 to ˜60 weight parts or from ˜45 to ˜55 weight parts of povidone; and from ˜2 to ˜8 weight parts or from ˜4 to ˜6 weight parts of a lubricant. A preferred lubricant is magnesium stearate.

Another aspect of the invention is an oral dosage form comprising micronized and non-micronized crystalline free-form metaxalone and one or more pharmaceutically acceptable excipients that maintains a stable dissolution profile over a period of at least 6 months when tested for 90 minutes at 1, 2, 3 and 6 month intervals under the dissolution testing described in USP, Metaxalone Tablets, comprising from 500 to 750, from 500 to 680, or 640 mg of said metaxalone. (Sec, https://dailymed.nlm.nih.gov/dailymed/drugInfo.cfm?setid=b0f165ff-027e-8a66-6083-6e808b838554).

In another embodiment, the w/w % of metaxalone released after 6 months differs by no more than ˜5 w/w %, ˜3 w/w %, or ˜2 w/w %, on an absolute basis, from the percentage of metaxalone released after 1, 2, 3 or 6 months, when stored at accelerated condition of 40° C. and 75% relative humidity and subjecting two or more of the plurality of tablets to a multiple time-point dissolution protocol comprising determining the amount of micronized and non-micronized crystalline free-form metaxalone in the tablets released at two or more time points from 30 to 60 minutes apart under dissolution testing described in USP, Metaxalone Tablets (January 2022).

In another embodiment, the hardness and particle size distribution of the micronized and non-micronized crystalline free-form metaxalone is provided producing a stable dissolution profile over a period of at least 6 months when tested for 90 minutes at 1, 2, 3 and 6 month intervals under the dissolution testing described in USP, Metaxalone Tablets, comprising from 500 to 750, from 500 to 680, or 640 mg of said metaxalone.

In another embodiment, the oral dosage form includes a means for maintaining a stable dissolution profile.

In another embodiment, the oral dosage form (e.g., tablet or capsule) ha a hardness in the range of ˜6 to ˜35 kp, ˜6 to ˜25 kp, or ˜6 to ˜17 kp.

In another embodiment, a dissolution specification for the oral dosage form is measured according to a first dissolution protocol.

In another embodiment, a testing of the oral dosage form is performed in accordance to said first dissolution protocol.

In another embodiment, the oral dosage form is tested for hardness that complies with the first dissolution specification (i.e., the first target hardness).

In another embodiment, the oral dosage form is compressed to a predetermined target hardness.

In another embodiment, the oral dosage form of metaxalone tablets are bioequivalent to SKELAXIN® 800 mg in fed and fasted states when administered to a patient. A plurality of tablets are subjected to a multiple time-point dissolution protocol comprising determining the amount of micronized and non-micronized metaxalone in the tablets are released at two or more time points from 30 to 60 minutes apart under dissolution testing described in USP, Metaxalone Tablets (January 2022).

In another embodiment, the micronized crystalline free-form metaxalone is characterized by a d50 in the range of ˜7 to ˜86 μm when tested according to USP <429> Laser Light Diffraction. (See, https://www.usp.org/sites/default/files/usp/document/harmonization/gen-chapter/g13_pf_35_3_2009.pdf)

In another embodiment, the non-micronized crystalline free-form metaxalone is characterized by a d50 in the range of ˜98 to ˜516 μm when tested according to USP <429> Laser Light Diffraction.

In another embodiment, the d50 of the non-micronized crystalline free-form metaxalone is at least ˜2, ˜10, ˜50 or ˜100 times greater than the d50 of the micronized crystalline free-form metaxalone when tested according to USP <429> Laser Light Diffraction.

In another embodiment, the plurality of tablets comprises greater than ˜10,000, ˜40,000, ˜100,000 or ˜1,000,000 tablets.

In another embodiment, the sum of the micronized crystalline free-form metaxalone and the non-micronized crystalline free-form metaxalone is in the range of ˜70 w/w % to ˜98 w/w % or ˜80 w/w % to ˜90 w/w % of the total weight of the tablet.

In another embodiment, the weight ratio of the micronized crystalline free-form metaxalone to the non-micronized crystalline free-form metaxalone is in the range of ˜10:1 to ˜1:10, ˜2:1 to ˜1:2, or ˜2:1 to ˜1:1.

In another embodiment, said pharmaceutically acceptable excipients include one or more of fillers, additives, diluents, binders, dyes, disintegrants, lubricants and other suitable excipients.

In another embodiment, the one or more excipients are in the range of ˜2 to ˜30 w/w % or ˜10 to ˜20 w/w % of the total tablet weight.

In another embodiment, at least ˜45 w/w % of the plurality of tablets are characterized by a hardness substantially equal to a predetermined hardness.

Another aspect of the invention is an immediate release oral dosage form of micronized and non-micronized crystalline free-form metaxalone and one or more pharmaceutically acceptable tableting excipients, whereby the oral dosage form maintains a stable dissolution profile over a period of 6 months or more when tested at 90 minutes under the dissolution testing described in USP, Metaxalone Tablets, comprising from 500 to 750, from 500 to 680, or 640 mg of said metaxalone.

In another embodiment, the percentage of micronized and non-micronized crystalline free-form metaxalone released after 6 month differs by no more than ˜5 w/w %, ˜3 w/w %, or ˜2 w/w %, on an absolute basis, from the percentage of said metaxalone released after 0 months or 3 months, when stored at accelerated condition of 40° C. and 75% relative humidity.

In another embodiment, the oral dosage form comprises micronized and non-micronized crystalline free-form metaxalone, propylene glycol alginate, and alginic acid, wherein said oral dosage form is free of oils, lipid-based excipients and co-solvents thereof.

In another embodiment, the oral dosage form comprises ˜640 mg of micronized and non-micronized crystalline free-form metaxalone.

In another embodiment, the oral dosage form comprises ˜40 to ˜60 weight parts povidone, ˜20 to ˜35 weight parts lactose monohydrate, ˜4 to ˜6 weight parts magnesium stearate, ˜10 to ˜30 weight parts propylene glycol alginate, and ˜10 to ˜30 weight parts alginic acid.

In another embodiment, the oral dosage form comprises ˜0.01 to ˜0.07 w/w % povidone, ˜0.02 to ˜0.05 w/w % lactose monohydrate, ˜0.005 to ˜0.007 w/w % magnesium stearate, ˜0.01 to ˜0.03 w/w % propylene glycol alginate, and ˜0.01 to ˜0/03 w/w % alginic acid.

In another embodiment, the weight ratio of micronized crystalline free-form metaxalone to non-micronized crystalline free-form metaxalone is ˜40:60 to ˜80:20.

In another embodiment, the oral dosage form comprises ˜90 w/w % of said micronized crystalline free-form metaxalone having a particle size in the range of ˜50 to ˜500 μm and ˜2 to ˜10 w/w % of said non-micronized crystalline free-form metaxalone is retained on a #30 Sieve.

In another embodiment, the oral dosage form comprises ˜10 to ˜30 weight parts propylene glycol alginate and ˜10 to ˜30 weight parts alginic acid.

In another embodiment, the oral dosage form comprises ˜90 w/w % of said micronized crystalline free-form metaxalone having a particle size less than ˜100 microns and at least ˜35 w/w % of said non-micronized crystalline free-form metaxalone being retained on a #120 Sieve.

In another embodiment, the oral dosage form comprises ˜90 w/w % of said micronized crystalline free-form metaxalone having a particle size less than ˜75 μm and less than ˜5 w/w % of said non-micronized crystalline free-form metaxalone being retained on a #30 Sieve.

In another embodiment, the oral dosage form comprises ˜10 w/w % of the micronized crystalline free-form metaxalone having a particle size of ˜0.81 microns, ˜50 w/w % of the micronized crystalline free-form metaxalone having a particle size of ˜10.11 microns, ˜90 w/w % of the micronized crystalline free-form metaxalone having a particle size of ˜49.91 microns, ˜1 w/w % of the non-micronized crystalline free-form metaxalone being retained on a #30 Sieve, ˜49 to ˜63 w/w % of the non-micronized crystalline free-form metaxalone being retained on a #120 Sieve, and ˜28 to ˜30 w/w % of the non-micronized crystalline free-form metaxalone being retained on a #325 Sieve.

In another embodiment, the oral dosage form comprises ˜5 to ˜15 w/w % of the micronized crystalline free-form metaxalone having a particle size in the range of ˜0.7 to ˜0.9 μm, ˜40 to ˜60 w/w % of the micronized crystalline free-form metaxalone having a particle size in the range of ˜5 to ˜15 μm, and ˜85 to ˜95 w/w % of the micronized crystalline free-form metaxalone having a particle size of ˜40 to ˜60 μm.

In another embodiment, the oral dosage form comprises ˜0.5 to ˜5 w/w % of the non-micronized crystalline free-form metaxalone being retained on a #30 Sieve, ˜40 to ˜70 w/w % of the non-micronized crystalline free-form metaxalone being retained on a #120 Sieve, and ˜20 to ˜40 w/w % of the non-micronized crystalline free-form metaxalone being retained on a #325 Sieve.

In another embodiment, the oral dosage form is made by the following steps or acts: preparing a granulating solution by dissolving povidone in purified water by mixing; pre-mixing micronized and non-micronized crystalline free-form metaxalone, propylene glycol alginate and alginic acid at a suitable head speed producing a pre-mix blend; wet granulating said pre-mix blend while adding said granulating solution to said pre-mix blend producing a wet granulation product; drying said wet granulation product to a predetermined moisture content producing a dried granulation product; commute milling said dried granulation product producing a milled product; mixing magnesium stearate into said milled product; and compressing said milled product producing said oral dosage form, wherein said oral dosage form is free of oils, lipid-based excipients and co-solvents thereof.

In another embodiment, the oral dosage form is free of lipid-based excipients including one or more of triglycerides, mixed glycerides, and polar oils; and co-solvents including one or more of ethanol, propylene glycol, glycerol, glycerides, glyceryls, glycerins, polyethylene glycols, and lipid-based synthetic surfactants.

In another embodiment, the oral dosage form comprises micronized and non-micronized crystalline free-form metaxalone, wherein said oral dosage form is free of oils, lipid-based excipients and co-solvents thereof.

Another aspect of the invention is a method of treating acute painful musculoskeletal conditions comprising administering an oral dosage form comprising micronized and non-micronized crystalline free-form metaxalone, wherein said oral dosage form is free of oils, lipid-based excipients and co-solvents thereof.

Another aspect of the invention is an oral dosage form comprising a micronized active pharmaceutical ingredient, and a non-micronized pharmaceutical ingredient, wherein said micronized and non-micronized active pharmaceutical ingredients are substantially insoluble in water.

Another aspect of the invention is an oral dosage form comprising a micronized active pharmaceutical ingredient, a non-micronized active pharmaceutical ingredient, propylene glycol alginate, and alginic acid, wherein said oral dosage form is free of oils, lipid-based excipients and co-solvents thereof, and wherein said micronized and non-micronized active pharmaceutical ingredients are substantially insoluble in water.

In another embodiment, the oral dosage form has a dissolution profile in accordance with 900 mL of fasting state Simulated Intestinal Fluid, USP Apparatus 2 Paddle at 50 rpm of ˜16.4 w/w % at 30 minutes, ˜37.2 w/w % at 60 minutes, ˜55.8 w/w % at 90 minutes, ˜67.1 w/w % at 120 minutes, ˜75.2 w/w % at 150 minutes, ˜81.1 w/w % at 180 minutes, ˜85.6 w/w % at 210 minutes, ˜88.5 w/w % at 240 minutes, ˜91.1 w/w % at 270 minutes and ˜93.0 w/w % at 300 minutes.

In another embodiment, the oral dosage form has a dissolution profile in accordance with 900 mL of fasting state Simulated Intestinal Fluid, USP Apparatus 2 Paddle at 50 rpm of ˜10.1 w/w % at 30 minutes, ˜30.1 w/w % at 60 minutes, ˜43.7 w/w % at 90 minutes, ˜53.5 w/w % at 120 minutes, ˜59.9 w/w % at 150 minutes, ˜65.7 w/w % at 180 minutes, ˜70.2 w/w % at 210 minutes, ˜74.4 w/w % at 240 minutes, ˜77.2 w/w % at 270 minutes and ˜80.2 w/w % at 300 minutes.

In another embodiment, the oral dosage form has a dissolution profile in accordance with 900 mL of fasting state Simulated Intestinal Fluid, USP Apparatus 2 Paddle at 50 rpm of ˜10.0 w/w % at 30 minutes, ˜31.8 w/w % at 60 minutes, ˜45.2 w/w % at 90 minutes, ˜55.6 w/w % at 120 minutes, ˜62.6 w/w % at 150 minutes, ˜67.7 w/w % at 180 minutes, ˜72.4 w/w % at 210 minutes, ˜75.9 w/w % at 240 minutes, ˜78.9 w/w % at 270 minutes and ˜81.4 w/w % at 300 minutes.

In another embodiment, the oral dosage form has a dissolution profile in accordance with 900 mL of fasting state Simulated Intestinal Fluid, USP Apparatus 2 Paddle at 50 rpm of ˜12.0 w/w % at 30 minutes, ˜36.9 w/w % at 60 minutes, ˜49.9 w/w % at 90 minutes, ˜59.4 w/w % at 120 minutes, ˜66.0 w/w % at 150 minutes, ˜71.8 w/w % at 180 minutes, ˜75.2 w/w % at 210 minutes, ˜78.9 w/w % at 240 minutes, ˜81.6 w/w % at 270 minutes and ˜83.7 w/w % at 300 minutes.

In another embodiment, the oral dosage form has a dissolution profile in accordance with 900 mL of fasting state Simulated Intestinal Fluid, USP Apparatus 2 Paddle at 50 rpm of ˜8.6 w/w % at 30 minutes, ˜17.4 w/w % at 60 minutes, ˜33.4 w/w % at 90 minutes, ˜47.0 w/w % at 120 minutes, ˜57.7 w/w % at 150 minutes, ˜65.4 w/w % at 180 minutes, ˜70.4 w/w % at 210 minutes, ˜74.6 w/w % at 240 minutes, ˜78.1 w/w % at 270 minutes and ˜81.4 w/w % at 300 minutes.

In another embodiment, the oral dosage form has a dissolution profile in accordance with 900 mL of fasting state Simulated Intestinal Fluid, USP Apparatus 2 Paddle at 50 rpm of ˜5.1 w/w % at 30 minutes, ˜12.7 w/w % at 60 minutes, ˜26.3 w/w % at 90 minutes, ˜42.7 w/w % at 120 minutes, ˜55.1 w/w % at 150 minutes, ˜63.4 w/w % at 180 minutes, ˜69.6 w/w % at 210 minutes, ˜74.6 w/w % at 240 minutes, ˜78.2 w/w % at 270 minutes and ˜81.7 w/w % at 300 minutes.

In another embodiment, the oral dosage form has a dissolution profile in accordance with 900 mL 0.5% SLS in water Apparatus II 100 rpm of ˜24-29 w/w % at 15 minutes, ˜56-64 w/w % at 30 minutes, ˜81-91 w/w % at 45 minutes, ˜90-99 w/w % at 60 minutes and ˜94-102 w/w % at 90 minutes.

In another embodiment, the oral dosage form has a dissolution profile in accordance with 900 mL 0.5% SLS in water Apparatus II 100 rpm of ˜10.1-12.8 w/w % at 15 minutes, ˜31.8-34.0 w/w % at 30 minutes, ˜50.0-52.3 w/w % at 45 minutes, ˜70.2-72.1 w/w % at 60 minutes and ˜90.1-93.0 w/w % at 90 minutes

In another embodiment, the oral dosage form has a dissolution profile in accordance with USP Apparatus II 100 rpm 37° C.±0.5° C. (n=3) 900 mL pH 4.5 acetate buffer of ˜4.6 w/w % average, ˜4.3-5.0 w/w % range and ˜7.6 w/w % RSD at 15 minutes; ˜11.3 w/w % average, ˜11.0-11.6 w/w % range and ˜2.9 w/w % RSD at 30 minutes; ˜19.7 w/w % average, ˜18.7-21.3 w/w % range and ˜6.9 w/w % RSD at 45 minutes; ˜27.9 w/w % average, ˜27.0-29.7 w/w % range and ˜5.3 w/w % RSD at 60 minutes; ˜40.0 w/w % average, ˜39.3-40.7 w/w % range and ˜1.8 w/w % RSD at 90 minutes; and ˜46.1 w/w % average, ˜46.0-46.3 w/w % and ˜0.3 w/w % RSD at 120 minutes.

In another embodiment, the oral dosage form has a dissolution profile in accordance with USP Apparatus II 100 rpm 37° C.±0.5° C. (n=3) 900 mL pH 6.8 acetate buffer of ˜3.1 w/w % average, ˜2.8-3.5 w/w % range and ˜11.7 w/w % RSD at 15 minutes; ˜6.6 w/w % average, ˜5.9-7.4 w/w % range and ˜11.5 w/w % RSD at 30 minutes; ˜11.2 w/w % average, ˜9.7-13.0 w/w % range and ˜15.0 w/w % RSD at 45 minutes; ˜15.0 w/w % average, ˜12.9-17.5 w/w % range and ˜15.4 w/w % RSD at 60 minutes; ˜23.1 w/w % average, ˜21.4-25.0 w/w % range and ˜7.9 w/w % RSD at 90 minutes; and ˜30.4 w/w % average, ˜29.3-32.2 w/w % and ˜5.1 w/w % RSD at 120 minutes.

In another embodiment, the oral dosage form yields Arithmetic Mean % CV pharmacokinetic parameters fasting median range for of Cₘₐₓ˜2153.22 ng/mL±˜59.22 ng/ml, Tₘₐₓ˜3.5 hr±˜1.50-12.00 hr, AUC˜15,723.65-16,023.38 ng-hr/ml±˜50.87-50.23 ng-hr/ml, T_{1/2}˜5.17 hr±˜41.41 hr, and KEL˜0.1534 l/hr±36.96 l/hr.

In another embodiment, the oral dosage form yields arithmetic mean % CV pharmacokinetic parameters fed median range of Cₘₐₓ˜2684.20 ng/ml±˜58.27 ng/ML, Tₘₐₓ˜8.00 hr±˜3.50-24.00 hr, AUC˜16,856.88-20,035.82 ng-hr/ml±˜43.58-51.81 ng-hr/ml, T_{1/2}˜2.07 hr±˜31.50 hr, and KEL˜0.3699 l/hr±33.01 l/hr.

In another embodiment, the oral dosage form yields Geometric Means Ratio of Means and 90% Confidence Intervals Ln-Transformed Data fed of AUC₀₋ₜ˜14,600.21 ng-hr/ml, AUC_{0-inf}˜14,840.39 ng-hr/ml, Cₘₐₓ 2207.56 ng/ml.

In another embodiment, the oral dosage form yields Geometric Means Ratio of Means and 90% Confidence Intervals Ln-Transformed Data fed of AUC₀₋ₜ˜13,686.84 ng-hr/ml, AUC_{0-inf}˜13,988.59 ng-hr/ml, Cₘₐₓ 1798.83 ng/ml.

In another embodiment, the oral dosage form consists essentially of 640 mg micronized crystalline free-form metaxalone and non-micronized crystalline free-form metaxalone, propylene glycol alginate and alginic acid, wherein said oral dosage form maintains a stable dissolution profile over 3 or 6 months.

In another embodiment, the oral dosage form consists essentially of ˜40 to ˜80 w/w % micronized crystalline free-form metaxalone and ˜20 to ˜60 w/w % non-micronized crystalline free-form metaxalone and one or more pharmaceutically acceptable excipients, wherein ˜90 w/w % of the micronized crystalline free-form metaxalone has a particle size less than ˜100 μm, wherein at least ˜35 w/w % of the non-micronized crystalline free form metaxalone are retained on a #120 Sieve, and wherein said oral dosage form maintains a stable dissolution profile over 3 or 6 months.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1 Representative Formulation

Table 1 describes a representative batch formulation for a 640 mg tablet of the current invention:

TABLE 1

| | | | | | | | Ex1d | |
|---|---|---|---|---|---|---|---|---|
| | | | | | | | Quantity/ | |
| | Ex1a | | Ex1b | | Ex1c | | Batch | |
| Ingredient | Quantity/Batch (kg) | | | | | | (kg) | |
| Metaxalone Micronized | 53.760 | kg | 41.472 | kg | 41.472 | kg | 53.760 | kg |
| Metaxalone | 35.840 | kg | 27.648 | kg | 27.648 | kg | 35.840 | kg |
| Lactose Monohydrate | 3.764 | kg | 2.903 | kg | 2.903 | kg | 3.764 | kg |
| FD&C Yellow #6 | 0.044 | kg | 0.035 | kg | 0.035 | kg | 0.044 | kg |
| Popylene Glycol Alginate | 2.240 | kg | 1.728 | kg | 1.728 | kg | 2.240 | kg |
| Alginic Acid | 2.240 | kg | 1.728 | kg | 1.728 | kg | 2.240 | kg |
| Providone | 6.720 | kg | 5.184 | kg | 5.184 | kg | 6.720 | kg |
| Purified Water | 22.8 | kg | 17.6 | kg | 17.6 | kg | ≈22.8 | kg |
| Magnesium Stearate | 0.672 | kg | 0.518 | kg | 0.518 | kg | 0.672 | kg |

| | | | | | | | |
|---|---|---|---|---|---|---|---|
| Total | 105.28 | kg | 81.216 | kg | 81.216 | 105.28 | kg |

### Example 2 Representative Metaxalone Particle Sizes

Tables 2a and 2b describes representative particle sizes for the micronized and non-micronized metaxalone particles used in the formulation of Table 1.

TABLE 2a

| | Particle Size Analysis by Malvern Method | | |
|---|---|---|---|
| | 10% of the | 50% of the | 90% of the |
| | Particles (d₁₀) | Particles (d₅₀) | Particles (d₉₀) |
| Lot2a | 0.81μ | 10.11μ | 49.91μ |

* Used in Ex1a, Ex1b, and Ex1c

TABLE 2b

| | Particle Size Analysis by Sieve Method | | |
|---|---|---|---|
| | % Retained on | | |
| | Sieve # 30 | Sieve # 120 | Sieve # 325 |
| Lot2b | 1% | 49% | 30% |
| Lot2c | 1% | 63% | 29% |
| Lot2d | 1% | 61% | 28% |

* Used in Ex1a, Ex1b, and Ex1c, respectively

### Example 3 Representative Manufacturing Method

This example includes detailed information describing the manner in which Metaxalone Tablets 640 mg are manufactured, using the formulation and metaxalone described in Tables 1 and 2.

Granulating Solution: Povidone was dissolved by slowly adding to Purified Water while mixing using mixer at required speed.

Pre-Mixing: Metaxalone Micronized, Metaxalone, FD&C Yellow #6, Propylene Glycol Alginate, and Alginic Acid were mixed at a suitable head speed.

Wet-Granulation: To the above Pre-Mix blend, added granulating solution while mixing at suitable Head Speed.

Drying: Wet-Granulation was dried Dryer to achieve desired moisture content. Milling: Upon completion of drying process, dried granulation was milled using commuting mill.

Final-Mixing: Magnesium Stearate was added to milled blend and lubricated using suitable blender.

Compression: Final-Mix Blend was compressed into tablets using Rotary Tablet Press.

### Example 4 Dissolution Test Results/Fasted State Simulated Intestinal Fluid

Tables 4a and 4b describes dissolution test results for 640 mg tablets produced by the method of Example 3 (except where otherwise noted) and 800 mg Skelaxin® tablets.

TABLE 4a

| Product Strength | | | | | | |
|---|---|---|---|---|---|---|
| Parts of Tablets equivalent to contain 100 mg drug | | | | | | |
| Diss. Method | | | | | | |
| 900 mL of Fasting State Simulated Intestinal Fluid, | | | | | | |
| USP Apparatus 2 (Paddle) at 50 rpm | | | | | | |
| | | Metaxalone | | | Ex4c | Ex4d |
| | | Tablets | | | (contains | (contains |
| | | Ex4a | | | 60% | 60% |
| | | (contains | Ex4b | | micronized | micronized |
| | | 100% | (contains 50% | | and 40% | and 40% |
| | | Micronized | micronized and | | coarse | coarse |
| Skelaxin Tablets | | Drug) | 50% coarse API) | | API) | API |
| Original Strength | | | | | | |
| 800 mg | | 640 mg | | | | |
| Number of Unit Tested | | | | | | |
| n = 3 | n = 2 | n = 3 | n = 3 | n = 3 | n = 3 | n = 3 |

% of Dissolution

| | | | | | | | |
|---|---|---|---|---|---|---|---|
| 30 | 19.7 | 19.3 | 16.4 | 10.1 | 10.0 | 12.0 | 8.6 |
| Minutes | | | | | | | |
| 60 | 28.2 | 27.9 | 37.2 | 30.1 | 31.8 | 36.9 | 17.4 |
| Minutes | | | | | | | |
| 90 | 34.3 | 34.6 | 55.8 | 43.7 | 45.2 | 49.9 | 33.4 |
| Minutes | | | | | | | |
| 120 | 38.7 | 39.6 | 67.1 | 53.5 | 55.6 | 59.4 | 47.0 |
| Minutes | | | | | | | |
| 150 | 43.3 | 44.0 | 75.2 | 59.9 | 62.6 | 66.0 | 57.7 |
| Minutes | | | | | | | |
| 180 | 47.2 | 47.5 | 81.1 | 65.7 | 67.7 | 71.8 | 65.4 |
| Minutes | | | | | | | |
| 210 | 50.8 | 51.1 | 85.6 | 70.2 | 72.4 | 75.2 | 70.4 |
| Minutes | | | | | | | |
| 240 | 53.7 | 54.2 | 88.5 | 74.4 | 75.9 | 78.9 | 74.6 |
| Minutes | | | | | | | |
| 270 | 56.0 | 56.9 | 91.1 | 77.2 | 78.9 | 81.6 | 78.1 |
| Minutes | | | | | | | |
| 300 | 58.3 | 59.4 | 93.0 | 80.2 | 81.4 | 83.7 | 81.4 |
| Minutes | | | | | | | |

TABLE 4b

| Product Strength | | |
|---|---|---|
| Parts of Tablets equivalent to contain 100 mg drug | | |
| Dissolution Method | | |
| 900 mL of Fasting State Simulated Intestinal Fluid, USP Apparatus 2 | | |
| (Paddle) at 50 rpm | | |
| Batch Description | | |
| | | Metaxalone Tablets |
| | | Ex1a |
| | | (contains 60% Micronized and 40% |
| Skelaxin Tablets | | Coarse Drug) |
| Original Strength | | |
| 800 mg | | 640 mg |
| Unit Tested | | |
| n = 3 | n = 2 | N = 6 |

% of Dissolution

| | | | | |
|---|---|---|---|---|
| 30 | Minutes | 19.7 | 19.3 | 5.1 |
| 60 | Minutes | 28.2 | 27.9 | 12.7 |
| 90 | Minutes | 34.3 | 34.6 | 26.3 |
| 120 | Minutes | 38.7 | 39.6 | 42.7 |
| 150 | Minutes | 43.3 | 44.0 | 55.1 |
| 180 | Minutes | 47.2 | 47.5 | 63.4 |
| 210 | Minutes | 50.8 | 51.1 | 69.6 |
| 240 | Minutes | 53.7 | 54.2 | 74.6 |
| 270 | Minutes | 56.0 | 56.9 | 78.2 |
| 300 | Minutes | 58.3 | 59.4 | 81.7 |

### Example 5 Dissolution Test Results/SLS and pH Buffers

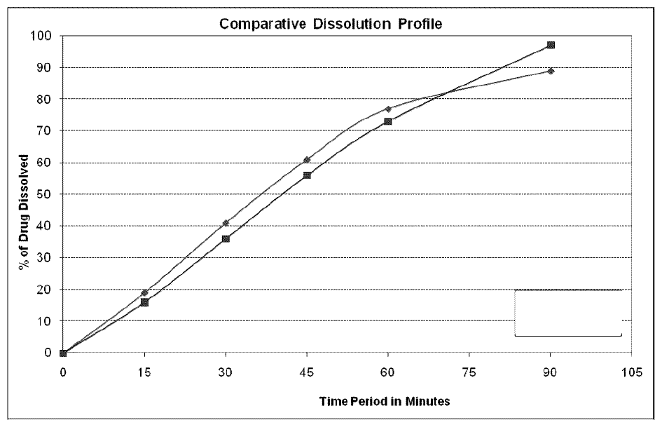

Tables 5a and 5b and FIG. 1 describe additional dissolution test results for 640 mg tablets produced by the method of Example 3 and 800 mg Skelaxin® tablets.

TABLE 5a

| Dissolution Conditions | | |
|---|---|---|
| 900 mL 0.5% SLS in Water, Apparatus II, 100 rpm | | |
| Product Description | | |
| | Metaxalone | Metaxalone |
| Skelaxin | Tablets 640 mg | Tablets 640 mg |
| Tablets 800 mg | Ex4a | Ex4d |

% of Drug Dissolved in

| | | | |
|---|---|---|---|
| 15 Minutes | 19 | 27 | 11.4 |
| | 17-21 | 24-29 | 10.1-12.8 |
| | 6.1 | 7.6 | 12.0 |
| 30 Minutes | 41 | 60 | 32.9 |
| | 36-44 | 56-64 | 31.8-34.0 |
| | 5.1 | 5.0 | 3.3 |
| 45 Minutes | 61 | 85 | 51.1 |
| | 60-64 | 81-91 | 50.0-52.3 |
| | 1.9 | 3.8 | 2.3 |
| 60 Minutes | 77 | 96 | 71.1 |
| | 75-79 | 90-99 | 70.2-72.1 |
| | 1.5 | 3.2 | 1.4 |
| 90 Minutes | 89 | 98 | 91.9 |
| | 87-90 | 94-102 | 90-1-93.0 |
| | 1.1 | 2.4 | 1.7 |

TABLE 5b

| | Dissolution | | | |
|---|---|---|---|---|
| | Test Product vs Skelaxin Tablets 800 mg | | | |
| | Method: USP Apparatus II, 100 rpm. 37° C. ± 0.5° C. (n = 3) | | | |
| | 900 mL pH 4.5 Acetate Buffer | | 900 mL pH 6.8 Phosphate Buffer | |
| Time Points | Ex1a | Skelaxin | Ex1a | Skelaxin |

% of Drug Dissolved in

| 15 | Minutes | | | | |
|---|---|---|---|---|---|
| Average (%) | | 4.6 | 8.0 | 3.1 | 6.7 |
| Range (%) | | 4.3-5.0 | 7.9-8.3 | 2.8-3.5 | 6.2-7.7 |
| RSD (%) | | 7.6 | 2.5 | 11.7 | 12.5 |
| 30 | Minutes | | | | |
| Average (%) | | 11.3 | 19.8 | 6.6 | 15.0 |
| Range (%) | | 11.0-11.6 | 19.4-20.4 | 5.9-7.4 | 14.3-15.8 |
| RSD (%) | | 2.9 | 2.7 | 11.5 | 4.9 |
| 45 | Minutes | | | | |
| Average (%) | | 19.7 | 30.0 | 11.2 | 24.6 |
| Range (%) | | 18.7-21.3 | 29.6-30.6 | 9.7-13.0 | 24.1-25.6 |
| RSD (%) | | 6.9 | 1.7 | 15.0 | 3.4 |
| 60 | Minutes | | | | |
| Average (%) | | 27.9 | 38.0 | 15.0 | 32.2 |
| Range (%) | | 27.0-29.7 | 37.8-38.4 | 12.9-17.5 | 32.2-32.4 |
| RSD (%) | | 5.3 | 0.8 | 15.4 | 0.4 |
| 90 | Minutes | | | | |
| Average (%) | | 40.0 | 44.5 | 23.1 | 41.7 |
| Range (%) | | 39.3-40.7 | 44.3-44.7 | 21.4-25.0 | 41.2-42.1 |
| RSD (%) | | 1.8 | 0.4 | 7.9 | 1.1 |
| 120 | Minutes | | | | |

| | | | | |
|---|---|---|---|---|
| Average (%) | 46.1 | 47.9 | 30.4 | 44.5 |
| Range (%) | 46.0-46.3 | 47.8-48.2 | 29.3-32.2 | 44.4-44.7 |
| RSD (%) | 0.3 | 0.5 | 5.1 | 0.3 |

### Example 6 Bioequivalence Test Results

A randomized, single-dose, four-way, open-label, crossover study fasted and fed study comparing 640 mg metaxalone tablets produced by the method of Example 3 was conducted with the reference listed drug, Skelaxin® Tablets, 800 mg, on 47 healthy adult volunteers (29 male, 18 female). The data of the 47 subjects who completed the fasted and fed studies were used in the calculations of pharmacokinetic results using SAS. The 90% confidence interval for the geometric mean test-to-reference area and peak concentration ratios were within the bioequivalence interval of 0.80-1.25. The 640 mg tablets were proven to be bioequivalent to Skelaxin® Tablets 800 mg under fasted and fed conditions.

Results of the testing are presented in Tables 6a and 6b.

TABLE 6a

| | Subjects | | | | | | |
|---|---|---|---|---|---|---|---|
| Treatments | No. (M/F) | Arithmetic Mean (% CV) Pharmacokinetic Parameters¹ | | | | | |
| (Dose, Dosage, | Type Age: | Median (Range) for Tₘₐₓ | | | | | |
| Form, Route) | Mean | Cₘₐₓ | Tₘₐₓ | AUC | AUC | T_{1/2} | KEL |
| [Product ID] | (Range) | (ng/mL) | (hr) | (ng-hr/ml) | (ng-hr/mL) | (hr) | (1/hr) |
| Test A: | 47 | 2153.22 | 3.50 | 15723.65 | 16023.38 | 5.17 | 0.1534 |
| Metaxalone | (29/18) | (59.22) | (1.50-12.00) | (50.87) | (50.23) | (41.41) | (36.96) |
| 640 mg | Healthy | 2684.20 | 8.00 | 16856.88 | 20035.82 | 2.07 | 0.3699 |
| Tablets, Oral | Volunteers | (58.27) | (3.50-24.00) | (51.81) | (43.58) | (31.50) | (33.01) |
| (Fasting) | 35.3 | 2030.99 | 3.50 | 15925.66 | 17838.87 | 7.04 | 0.1184 |
| Test B: | (18-69) | (59.95) | (2.00-6.00) | (51.29) | (50.01) | (41.77) | (47.85) |
| Metaxalone | | 3763.86 | 5.00 | 22381.70 | 22959.73 | 4.59 | 0.1804 |
| 640 mg | | (58.84) | (2.50-24.00) | (51.27) | (50.83) | (43.72) | (45.15) |
| Tablets, Oral | | | | | | | |
| (Fed) | | | | | | | |
| Reference C: | | | | | | | |
| SKELAXIN ® | | | | | | | |
| 800 mg | | | | | | | |
| Tablets, Oral | | | | | | | |
| (Fasting) | | | | | | | |
| Reference D: | | | | | | | |
| SKELAXIN ® | | | | | | | |
| 800 mg | | | | | | | |
| Tablets, Oral | | | | | | | |
| (Fed) | | | | | | | |

TABLE 6b
Metaxalone
640 mg (1 × 640 mg)
Geometric Means, Ratio of Means, and 90% Confidence Intervals
Ln- Transformed Data
Bioequivalence Study
Test Product B - Fed (640 mg) vs. Test Product A - Fasting (640 mg)
N = 47

| Parameter | Test B | Test A | % Ratio | 90% C.I. |
|---|---|---|---|---|
| AUC₀₋ₜ | 14600.21 | 13686.84 | 106.67 | (98.35, 115.70) |
| AUC_{0-inf} | 14840.39 | 13988.59 | 106.09 | 98.23, 114.57 |
| Cₘₐₓ | 2207.56 | 1798.83 | 122.72 | 104.93, 143.53 |

Bioequivalence Study
Test Product B - Fed (640 mg) vs. Reference Product C - Fasting (800 mg)
N = 47

| Parameter | Test A | Reference C | % Ratio | 90% C.I. |
|---|---|---|---|---|
| AUC₀₋ₜ | 13686.84 | 13907.27 | 98.41 | (90.74, 106.74) |
| AUC_{0-inf} | 13988.59 | 14866.84 | 94.09 | (87.12, 101.62) |
| Cₘₐₓ | 1798.83 | 1735.28 | 103.66 | 88.64, 121.24 |

Bioequivalence Study
Test Product B - Fed (640 mg) vs. Reference Product D - Fed (800 mg)
N = 47

| Parameter | Test B | Reference D | % Ratio | 90% C.I. |
|---|---|---|---|---|
| AUC₀₋ₜ | 14600.21 | 19359.95 | 75.41 | (69.53, 81.80) |
| AUC_{0-inf} | 14840.39 | 19624.22 | 75.62 | (70.02, 81.67) |
| Cₘₐₓ | 2207.56 | 3046.51 | 72.46 | 61.96, 84.75) |

Bioequivalence Study
Reference Product D - Fed (800 mg) vs.
Reference Product C - Fasting (800 mg)
N = 47

| Parameter | Reference D | Reference C | % Ratio | 90% C.I. |
|---|---|---|---|---|
| AUC₀₋ₜ | 19359.95 | 13907.27 | 139.21 | (128.35, 150.99) |
| AUC_{0-inf} | 19624.22 | 14866.84 | 132.00 | (122.22, 142.56) |
| Cₘₐₓ | 3046.51 | 1735.28 | 175.56 | (150.11, 205.33) |

TABLE 7

| TEST | LIMITS/ | | | | | | | |
|---|---|---|---|---|---|---|---|---|
| INTERVAL | LABEL | INITIAL | 1M | 2M | 3M | 6M | 9M | 12M |
| Temp. | | N/A | 23.9-35.8 | 23.9-35.8 | 23.9-35.8 | 23.9-35.8 | 23.9-35.8 | 23.5-35.8 |
| Range | | | 24.1-26.2 | 24.1-26.4 | 24.1-26.4 | 24.1-26.4 | 23.5-27.4 | 23.5-27.4 |
| (° C.) | | | 24.0-35.6 | 23.9-35.8 | 23.9-35.8 | 23.9-35.8 | 23.9-35.8 | 23.5-35.8 |
| Humidity | | N/A | 55.2-68.8 | 55.2-68.8 | 55.2-68.8 | 55.2-68.8 | 55.2-68.8 | 55.2-68.8 |
| Range | | | 58.7-63.3 | 55.5-63.3 | 55.5-63.3 | 55.5-63.3 | 55.5-6.1 | 55.5-64.1 |
| (% RH) | | | 55.1-65.0 | 55.1-68.8 | 55.1-68.8 | 55.1-68.8 | 55.1-68.8 | 55.1-68.8 |
| Water | NMT 2.5% | Seg_{A} = | 0.9%, 1.1% | 1.0%, 1.1% | 1.0%, 1.1% | 1.0%, 1.2% | 1.0%, 1.3% | 1.0%, 1.3% |
| Content | | 1.0%, 1.4% | | | | | | |
| | | Seg_{B} = | | | | | | |
| | | 0.9%, 1.4% | | | | | | |
| | | Seg_{C} = | | | | | | |
| | | 0.9%, 1.3% | | | | | | |

| | LABEL/ | | | | | | | |
|---|---|---|---|---|---|---|---|---|
| TEST | LIMITS | INITIAL | 1M | 2M | 3M | 6M | 9M | 12M |
| Dissolution | 30 min | Seg_{A} | x = 31%, | x = 34%, | x = 34%, | x = 33%, | x = 31%, | x = 34%, |
| | 26-46% | x = 31%, 38% | 36% | 40% | 39% | 43% | 37% | 38% |
| | 90 mins | D₁ = | D₁ = | D₁ = | D₁ = | D₁ = | D₁ = | D₁ = |
| | Q = 80% | 29%, 39% | 32% 36% | 33%, 38% | 34%, 37% | 33%, 42% | 30%, 40% | 33%, 39% |
| | | D₂ = | D₂ = | D₂ = | D₂ = | D₂ = | D₂ = | D₂ = |
| | | 31%, 39% | 30%, 36% | 36%, 37% | 33%, 40% | 32%, 41% | 28%, 34% | 36%, 35% |
| | | D₃ = | D₃ = | D₃ = | D₃ = | D₃ = | D₃ = | D₃ = |
| | | 30%, 36% | 32%, 36% | 34%, 43% | 35%, 42% | 32%, 42% | 35%, 40% | 38%, 36% |
| | | D₄ = | D₄ = | D₄ = | D₄ = | D₄ = | D₄ = | D₄ = |
| | | 31%, 36% | 32%, 35% | 33%, 40% | 33%, 38% | 32%, 43% | 30%, 36% | 33%, 41% |
| | | D₅ = | D₅ = | D₅ = | D₅ = | D₅ = | D₅ = | D₅ = |
| | | 32%, 40% | 30%, 37% | 37%, 41% | 34%, 38% | 31%, 43% | 31%, 34% | 29%, 37% |
| | | D₆ = | D₆ = | D₆ = | D₆ = | D₆ = | D₆ = | D₆ = |
| | | 31%, 38% | 31%, 37% | 33%, 40% | 34%, 41% | 36%, 44% | 30%, 36% | 32%, 40% |
| | | Seg_{B} | x = 95%, 96% | x = 97%, 97% | x = 97%, 97% | x = 96%, 98% | x = 95% | x = 97%, 99% |
| | | x = 33%, 37% | D₁ = | D₁ = | D₁ = | D₁ = | D₁ = 94% | D₁ = |
| | | D₁ = | 96%, 96% | 96%, 96% | 97%, 97% | 96%, 98% | D₂ = 93% | 97%, 99% |
| | | 32%, 34% | D₂ = | D₂ = | D₂ = | D₂ = | D₃ = 96% | D₂ = |
| | | D₂ = | 94%, 96% | 97%, 96% | 98%, 98% | 96%, 97% | D₄ = 95% | 97%, 97% |
| | | 34%, 37% | D₃ = | D₃ = | D₃ = | D₃ = | D₅ = 95% | D₃ = |
| | | D₃ = | 95%, 96% | 97%, 97% | 97%, 98% | 96% 97% | D₆ = 95% | 98%, 98% |
| | | 31%, 38% | D₄ = | D₄ = | D₄ = | D₄ = | | D₄ = |
| | | D₄ = | 95%, 96% | 97%, 97% | 96%, 97% | 97%, 98% | | 97%, 99% |
| | | 31%, 36% | D₅ = | D₅ = | D₅ = | D₅ = | | D₅ = |
| | | D₅ = | 93%, 97% | 97%, 97% | 96%, 97% | 96%, 98% | | 94%, 100% |
| | | 33%, 41% | D₆ = | D₆ = | D₆ = | D₆ = | | D₆ = |
| | | D₆ = | 95%, 97% | 97%, 97% | 97%, 97% | 97%, 98% | | 96%, 99% |
| | | 34%, 37% | | | | | | |
| | | Seg_{C} | | | | | | |
| | | x = 34%, 35% | | | | | | |
| | | D₁ = | | | | | | |
| | | 33%, 35% | | | | | | |
| | | D₂ = | | | | | | |
| | | 36%, 34% | | | | | | |
| | | D₃ = | | | | | | |
| | | 32%, 34% | | | | | | |
| | | D₄ = | | | | | | |
| | | 35%, 36% | | | | | | |
| | | D₅ = | | | | | | |
| | | 34%, 34% | | | | | | |
| | | D₆ = | | | | | | |
| | | 33%, 36% | | | | | | |
| | | Seg_{A} | | | | | | |
| | | x = 94%, 97% | | | | | | |
| | | D₁ = | | | | | | |
| | | 94%, 97% | | | | | | |
| | | D₂ = | | | | | | |
| | | 94%, 97% | | | | | | |
| | | D₃ = | | | | | | |
| | | 93%, 96% | | | | | | |
| | | D₄ = | | | | | | |
| | | 94% 96% | | | | | | |
| | | D₅ = | | | | | | |
| | | 94% 97% | | | | | | |
| | | D₆ = | | | | | | |
| | | 93% 96% | | | | | | |
| | | Seg_{B} | | | | | | |
| | | x = 96%, 96% | | | | | | |
| | | D₁ = | | | | | | |
| | | 96% 95% | | | | | | |
| | | D₂ = | | | | | | |
| | | 96%, 96% | | | | | | |
| | | D₃ = | | | | | | |
| | | 95%, 96% | | | | | | |
| | | D₄ = | | | | | | |
| | | 95%, 95% | | | | | | |
| | | D₅ = | | | | | | |
| | | 96%, 97% | | | | | | |
| | | D₆ = | | | | | | |
| | | 96%, 96% | | | | | | |
| | | Seg_{C} | | | | | | |
| | | x = 96%, 95% | | | | | | |
| | | D₁ = | | | | | | |
| | | 96%, 95% | | | | | | |
| | | D₂ = | | | | | | |
| | | 97%, 94% | | | | | | |
| | | D₃ = | | | | | | |
| | | 96%, 95% | | | | | | |
| | | D₄ = | | | | | | |
| | | 96%, 95% | | | | | | |
| | | D₅ = | | | | | | |
| | | 96%, 95% | | | | | | |
| | | D₆ = | | | | | | |
| | | 94% 96% | | | | | | |
| ASSAY | 300 mg | x = 101.1% | x = 100.3% | x = 99.4% | x = 99.6% | x = 100.9% | x = 100.5% | x = 100.3% |
| | LIMITS | Seg_{A} | A₁ = 99.4% | A₁ = 99.5% | A₁ = 99.4% | A₁ = 101.0% | A₁ = 100.5% | A₁ = 100.3% |
| | 95.0%- | A₁ = 100.7% | A₂ = 101.2% | A₂ = 99.3% | A₂ = 99.7% | A₂ = 100.7% | A₂ = 100.4% | A₂ = 100.4% |
| | 105.0% | A₂ = 101.8% | | | | | | |
| | | Seg_{B} | | | | | | |
| | | A₁ = 101.2% | | | | | | |
| | | A₂ = 101.1% | | | | | | |
| | | Seg_{C} | | | | | | |
| | | A₁ = 100.9% | | | | | | |
| | | A₂ = 101.8% | | | | | | |
| | 300 mg | x = 101.4% | x = 99.7% | x = 99.6% | x = 98.8% | x = 100.4% | x = 99.6% | x = 100.3% |
| | 95.0%- | Seg_{A} | A₁ = 99.7% | A₁ = 99.4% | A₁ = 99.0% | A₁ = 100.6% | A₁ = 99.5% | A₁ = 100.3% |
| | 105.0% | A₁ = 100.0% | A₂ = 99.8% | A₂ = 99.8% | A₂ = 98.6% | A₂ = 100.2% | A₂ = 99.6% | A₂ = 100.2% |
| | | A₂ = 101.0% | | | | | | |
| | | Seg_{B} | | | | | | |
| | | A₁ = 100.8% | | | | | | |
| | | A₂ = 101.1% | | | | | | |
| | | Seg_{C} | | | | | | |
| | | A₁ = 100.4% | | | | | | |
| | | A₂ = 101.4% | | | | | | |

Table 7 shows stability data for compressed tablet containing micronized and non-micronized Metaxalone Tablets, 640 mg in accordance with the present invention.
Storage conditions: ICH CRT 25° C.±2° C., 60%+5% Relative Humidity
Manufacturing Site: 2090 Marietta Blvd., NW Atlanta GA 30318
Packaging Site: 1750 Chattahoochee Ave, NW Atlanta GA 30318
Test Facility: Mikart, LLC
Package Size: 100's
Mfg Lot: J210260
Pkg Lot: J210260A
Mfg Date: Oct. 28, 2021, Dec. 8, 2021
Pkg Date: Nov. 19, 2021, Dec. 16, 2021
Stab Date: Nov. 23, 2021, Dec. 20, 2021
Batch Type: Production
Batch Size: 140,000
Storage POS: N/A
Description: Peach colored, oval shaped compresses tablets. Debossed with “M640” on one side and plain on the other side. [symbol]
Container Desc: #9473, 38/400 150 cc White HDPE round wide mouth bottle
Container Mfr: Berry
Container Resin: Equistar LR7340-45
Container Desc: #9475, 38/400 white polypropylene CRC cap with SG-75 liner
Closure Mfr: Mold-Rite
Per compressed tablet formulation (M640):

| | | |
|---|---|---|
| Metaxalone USP (micronized)* | 384.00 | mg |
| Metaxalone USP (non-micronized)* | 256.00 | mg |
| Povidone USP K Value Range 29-32 | 48.00 | mg |
| Lactose Monohydrate NF | 26.88 | mg |
| FD&C Yellow #6 Aluminum Lake HT 15%-18% Pure Dye | 0.32 | mg |
| Propylene Glycol Alginate NF | 16.00 | mg |
| Magnesium Stearate NF | 16.00 | mg |
| Purified Water USP* | 4.80 | mg |

*Manufactured by Angelini Spa Italy. Purified water removed during processing.

### SUMMARY OF RESULTS

Package Integrity and Appearance at each time interval (1 Month to 12 Month): Bottle and cap free from visible defects and liner intact. Peach colored, oval shaped compressed tablets. Debossed with “M640” on one side and plain on the other side. Free from any visible defects.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An oral dosage form comprising ˜640 mg micronized and non-micronized crystalline free-form metaxalone and one or more pharmaceutically acceptable excipients, wherein said micronized crystalline free-form metaxalone comprises ˜40 to ˜80 w/w % of the oral dosage form,
wherein said non-micronized crystalline free-form metaxalone comprises ˜20 to ˜60 w/w % of the oral dosage form,
wherein said one or more pharmaceutically acceptable excipient comprises ˜10 to ˜30 weight parts propylene glycol alginate,
wherein ˜90 w/w % of said micronized micronized crystalline free-form metaxalone has a particle size less than 100 μm,
wherein at least ˜35 w/w % of said non-micronized crystalline free-form metaxalone is retained on a #120 Sieve, and
wherein said oral dosage form maintains a stable dissolution profile over 3 or 6 months.

2. The oral dosage form of claim 1, wherein the weight percentage of micronized and non-micronized crystalline free-form metaxalone released after 6 months differs by no more than ˜2 to ˜5 w/w % on an absolute basis from the weight percentage of micronized and non-micronized crystalline free-form metaxalone released after 0 to 3 months when stored at accelerated condition of 40° C. and 75% relative humidity.

3. The oral dosage form of claim 1, having a hardness in the range of ˜6 to ˜35 kp.

4. The oral dosage form of claim 1, having a hardness in the range of ˜6 to ˜25 kp.

5. The oral dosage form of claim 1, having a hardness in the range of ˜6 to ˜17 kp.

6. The oral dosage form of claim 1, having no food effect.

7. The oral dosage form of claim 1, having a predetermined hardness.

8. The oral dosage form of claim 1, wherein said micronized crystalline free-form metaxalone has a particle size in the range of ˜7 to ˜86 μm.

9-38. (canceled)

39. An oral dosage form comprising micronized free-form metaxalone and non-micronized free-form metaxalone and one or more pharmaceutically acceptable excipients, wherein said oral dosage form maintains a stable dissolution profile over 3 or 6 months.

40. The oral dosage form of claim 39, wherein the weight percentage of micronized and non-micronized crystalline free-form metaxalone released after 6 months differs by no more than ˜2 to ˜5 w/w % on an absolute basis from the weight percentage of micronized and non-micronized crystalline free-form metaxalone released after 0 to 3 months when stored at accelerated condition of 40° C. and 75% relative humidity.

41. The oral dosage form of claim 39, having a hardness in the range of ˜6 to ˜35 kp.

42. The oral dosage form of claim 39, having a hardness in the range of ˜6 to ˜25 kp.

43. The oral dosage form of claim 39, having a hardness in the range of ˜6 to ˜17 kp.

44. The oral dosage form of claim 39, having no food effect.

45. An oral dosage form comprising 640 mg micronized free-form metaxalone and non-micronized free-form metaxalone and one or more pharmaceutically acceptable excipients, wherein said oral dosage form maintains a stable dissolution profile over 3 or 6 months.

46. The oral dosage form of claim 45, wherein the weight percentage of micronized and non-micronized crystalline free-form metaxalone released after 6 months differs by no more than ˜2 to ˜5 w/w % on an absolute basis from the weight percentage of micronized and non-micronized crystalline free-form metaxalone released after 0 to 3 months when stored at accelerated condition of 40° C. and 75% relative humidity.

47. The oral dosage form of claim 45, having a hardness in the range of ˜6 to ˜35 kp.

48. The oral dosage form of claim 45, having a hardness in the range of ˜6 to ˜25 kp.

49. The oral dosage form of claim 45, having a hardness in the range of ˜6 to ˜17 kp.

50. The oral dosage form of claim 45, having no food effect.
